(19) United States
(12) Patent Application Publication
(10) Pub. No.: US 2026/0263081 A1
(43) Pub. Date: Sep. 10, 2026

(54) LIGATION CLIP WITH ELASTIC INSERT

(71) Applicant: TELEFLEX MEDICAL LLC, Morrisville, NC (US)

(72) Inventor: Adam R. Dunki-Jacobs, Cincinnati, OH (US)

(21) Appl. No.: 19/559,422

(22) Filed: Mar. 6, 2026

Related U.S. Application Data
(60) Provisional application No. 63/768,560, filed on Mar. 7, 2025.

(51) Int. Cl. A61B 17/122

(52) U.S. Cl. CPC A61B 17/1227

(57) ABSTRACT

A surgical clip is provided that has a first leg member including a first proximal portion, a first distal portion, and a first inner surface. The surgical clip also has a second leg member including a second proximal portion, a second distal portion, and a second inner surface. The surgical clip includes an elastic insert having a first limb attached to the first leg member, a second limb attached to the second leg member, and a hinge portion that pivotably biases the first and second leg members toward an open configuration. The hinge portion prevents or resists stress relaxation when the first and second leg members are in a closed or partially closed position in which the hinge portion is compressed.

### CROSS-REFERENCE TO RELATED APPLICATION

This application claims the benefit of priority to U.S. Provisional Patent Application No. 63/768,560 filed on Mar. 7, 2025, the disclosure of which is hereby incorporated by reference in its entirety.

### TECHNICAL FIELD

The present invention relates generally to surgical clips for ligation of tissue, and more particularly, to polymer surgical clips having an elastic insert.

### BACKGROUND

Ligation of tissue (e.g., blood vessels, lymph nodes, nerves, cystic ducts, and cardiac tissue) is a common practice for many surgical procedures. This may be performed by closing the vessel with a surgical clip or by suturing the vessel with the surgical thread. The use of surgical thread requires complex manipulations of a needle and surgical thread to form knots required to secure the vessel. Such complex manipulations are time consuming and difficult to perform, particularly in endoscopic surgical procedures characterized by limited space and/or visibility. In contrast, surgical clips are relatively quick and easy to apply.

Accordingly, the use of surgical clips in endoscopic and open surgical procedures has grown dramatically.

In multi-feed clip applier devices, such as the multi-fire polymer clip applier 10 illustrated in its fully open configuration in FIG. 1 disclosed in U.S. Pat. No. 7,585,304 incorporated by reference in its entirety, multiple polymer clips 13 are stored in the shaft 11 of the device. Over long periods of time, stress relaxation can occur that limits how much “spring back” occurs when the clip 13 is transferred from the shaft 11 of the applier 10 to the end effector jaws 12. This phenomenon requires the multi-feed clip applier devices 10 to open the clip 13 during loading. This is typically accomplished by pushing the clip 13 from behind with the jaws 12 of the clip applier closed, which then allows additional force applied at the back of the clip to open the spring-loaded jaws 12 of the applier 10. Such surgical clips 13 are often preloaded and stored within surgical clip appliers 10 prior to use in a partially closed configuration.

Over time, in this partially closed configuration, the surgical clips often begin to deform and do not fully rebound to their original state. This is especially problematic with polymer clips since the distance between the clip legs can change when stored for periods of time in a partially or fully compressed state. For instance, when initially manufactured or molded, the distance between a boss on a first leg of polymer clip and a boss on a second leg of the polymer clip is larger than the distance between the bosses of each leg after the clip has been stored in a partially or fully compressed position. This deformation creates issues when loading the surgical clips into the jaws of a preloaded clip applier, because successful loading requires the surgical clip to create a biasing force against the jaws of the clip applier to remain in place. Otherwise, the surgical clip will fall out of the jaws of the clip applier and will not be able to be used. Some clip appliers are able to overcome this limitation of the surgical clips by closing the jaws of the clip applier during clip loading, and then applying a force from behind the hinge of the surgical clip to ensure the surgical clip opens with the jaws of the device and does not fall out. However, closing the jaws of the clip applier during clip loading is not ideal for surgeons because it requires them to move the clip applier away from the vessel to reload the next clip.

Thus, there is a substantial need for an improved polymer ligating clip that is resistant to stress deformation in the hinge portion, or in other words, taking a set. Stated another way, there is a long felt need for a polymer ligating clip for which taking a set is prevented or minimized, while maintaining the original boss-to-boss distance of the clip, even after being stored in a compressed state within the shaft of a clip applier for a period of time, which may be as long as the shelf life of the applier and/or clip.

### BRIEF DESCRIPTION OF THE DRAWINGS

In order that the invention may be readily understood, aspects of this invention are illustrated by way of examples in the accompanying drawings.

FIG. 1 illustrates a multi-fire polymer clip applier which may be used with the surgical clip of the present disclosure.

FIG. 2 illustrates a perspective view of an exemplary embodiment of a surgical clip in an open configuration.

FIG. 3 illustrates a frontal view of the surgical clip of FIG. 2.

FIG. 4 illustrates a side view of the surgical clip of FIG. 2.

FIG. 5A illustrates an exemplary cutaway view of a first leg member of the surgical clip of FIG. 2.

FIG. SB illustrates an exemplary cross-section of the cutaway view of FIG. 5A.

FIG. 5C illustrates an exemplary cutaway view of a second leg member of the surgical clip of FIG. 2.

FIG. 5D illustrates an exemplary cross-section of the cutaway view of FIG. 5C.

FIG. 6 illustrates a perspective view of the surgical clip in the open configuration, further depicting an insert disposed within leg members of the clip.

FIG. 7 illustrates a side view of the embodiment of the elastic insert of the surgical clip in the open configuration of FIG. 2.

FIG. 8 illustrates a side view of an embodiment of the elastic insert of the surgical clip in the open configuration.

FIG. 9 illustrates a side view of an embodiment of the elastic insert of the surgical clip in the open configuration.

FIG. 10 illustrates a side view of an embodiment of the elastic insert of the surgical clip in the open configuration.

FIG. 11 illustrates a side view of an embodiment of the elastic insert of the surgical clip in the open configuration.

FIG. 12 illustrates a perspective view of an embodiment of the elastic insert of the surgical clip in the open configuration.

FIG. 13 illustrates a perspective view of an embodiment of the elastic insert of the surgical clip in the open configuration.

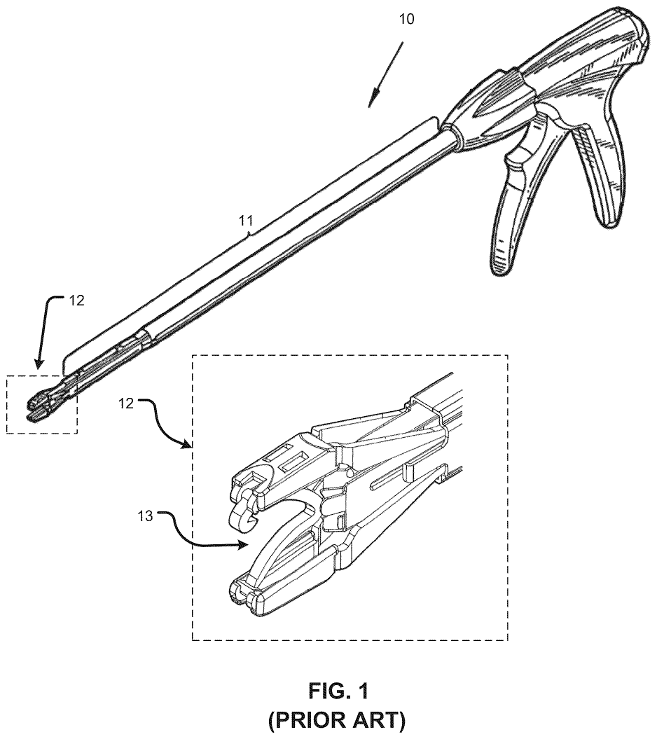

### DETAILED DESCRIPTION

The invention will now be described with reference to the figures, in which like reference numerals may refer to like parts throughout. The present invention is generally directed to a surgical clip configured to compress and/or ligate tissue (e.g., blood vessels, lymph nodes, nerves, cystic tubes, or cardiac tissue). The surgical clip may provide elongated leg members to increase the tissue-retaining capacity. For example, the surgical clip may be sized to fit in a 5 mm clip applier, but have an increased capacity compared to other 5 mm clips. To reinforce and stabilize at least one of the longer leg members, the surgical clip may have at least one lateral protrusion or wing member extending along at least one of the lateral sides of the leg member. The lateral protrusions may provide the leg member with an increased aspect ratio (width/thickness). Thus, the surgical clip is relatively wider along a portion of the leg member to increase the stiffness along at least a portion of the length of the leg member.

The surgical clip may also have teeth that receive a tissue clamping inner surface therebetween for increased axial security of tissue. The surgical clip may further have features that retain tissue proximate the hinge as the surgical clip closes. The surgical clip may also have rounded leg and/or hinge profiles enhancing applier jaw strength and facilitating easier loading from a cartridge. In some embodiments, the surgical clip may have an improved hinge member. For example, the hinge member may be improved by making the hinge out of a material with a yield strength and/or an allowable strain greater than that of the first leg member and/or the second leg member. According to some aspects, the hinge may include a metal insert that forms a substrate for an overmolding process, where the first leg member and/or the second leg member are overmolded onto the metal insert. Further, the metal insert may include at least one mechanical attachment configured to secure the metal insert to the first leg member and/or the second leg member. The hinge member may have a reduced thickness that improves the performance of the surgical clip during closure, i.e., by helping to stabilize the surgical clip in the jaws of a clip applier if the clip applier has mating features.

In accordance with conventional practice, as used herein, and unless otherwise indicated herein, the term “longitudinal” is directed to the dimension which extends along the length of the surgical clip and/or leg members from their respective proximal portions to their respective distal portions, as would be commonly understood by one of skill in the art.

Accordingly, the term “length” refers to a dimension of the surgical clip and/or one or more components along its longitudinal direction. Furthermore, the “transverse” direction is directed to any axis or direction which is orthogonal to the longitudinal lengths of the surgical clip or leg members. The term “vertical” refers to a dimension of the surgical clip and/or one or more components along a compression axis of the leg members. The term “thickness” refers to the dimension between opposing edges of the surgical clip and/or one or more components along the compression or vertical direction. The term “width” refers to a dimension of the surgical clip and/or one or more components in a lateral direction substantially transverse to the length and the thickness. The term “concave” and “convex” refers to the curvature of a surface or component visible when viewing an exterior of the surface or component. Similar terminology is used throughout the written disclosure, unless otherwise indicated.

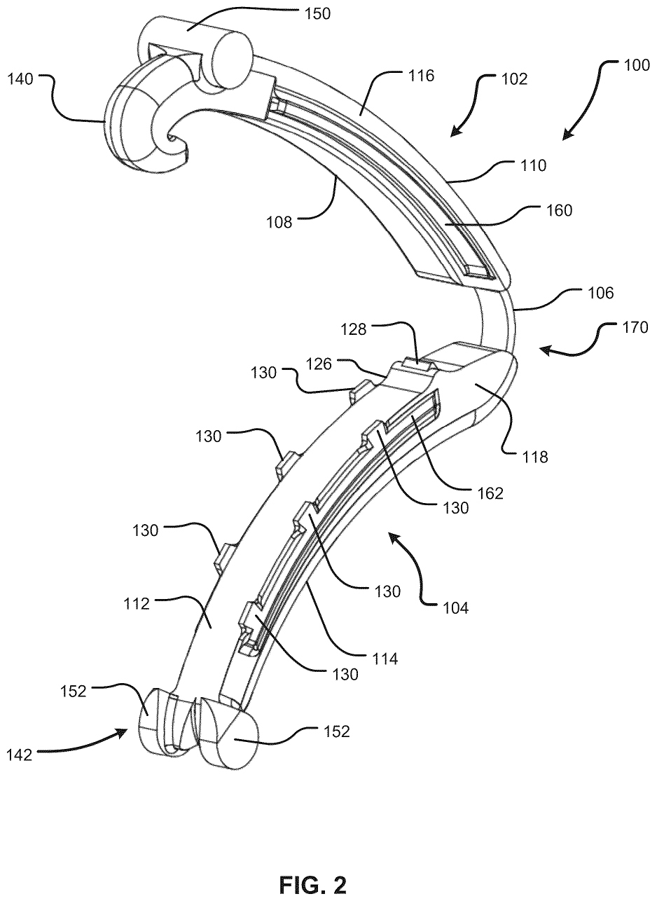

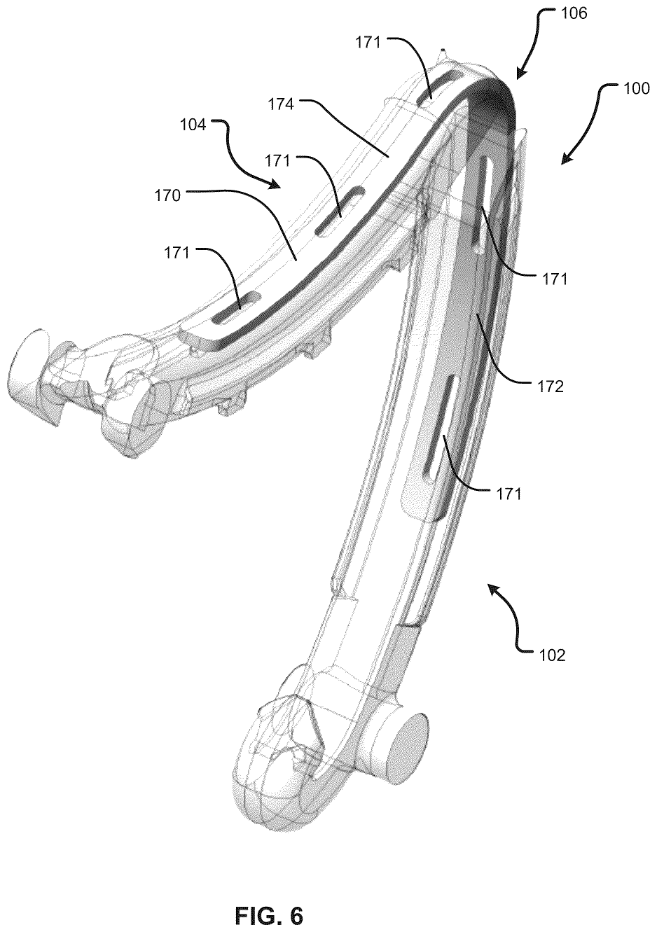

FIGS. 2-7 illustrate an embodiment of a surgical clip 100 of the present invention. The surgical clip 100 may include a first leg member 102 having a proximal portion and a distal portion, and a second leg member 104 having a proximal portion and a distal portion. The first and second leg members 102, 104 may be joined by an elastic insert 170 comprising a hinge member 106.

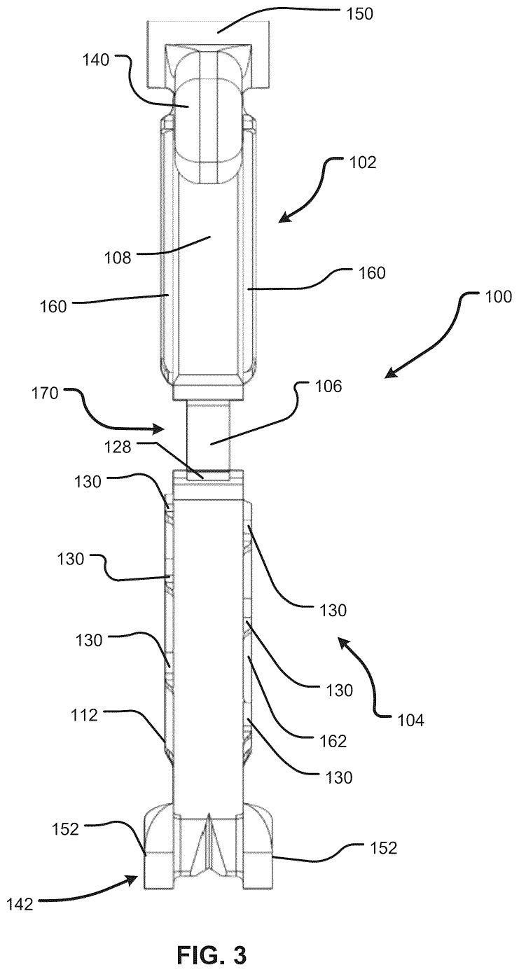

The first and second leg members 102, 104 may be curved along their lengths and include curved surfaces. For example, the first leg member 102 may include a first inner surface 108 and a first outer surface 110, and the second leg member 104 may include a second inner surface 112 and a second outer surface 114. The first inner surface 108 and first outer surface 110 may extend laterally between first side surfaces 116, and the second inner surface 112 and second outer surface 114 may extend laterally between second side surfaces 118. As shown in FIGS. 2 and 3, the first inner surface 108 may have a concave curvature, and the first outer surface 110 may have a convex curvature, each along the length of the first leg member 102. The second inner surface 112 may have a convex curvature, and the second outer surface 114 may have a concave curvature, each along the length of the second leg member 104. The concave curvature of the first inner surface 108 and/or the convex curvature of the first outer surface 110 may extend from the respective proximal portion to the respective distal portion, substantially the entire length of the first leg member 102. The convex curvature of the second inner surface 112 and/or the concave curvature of the second outer surface 114 may extend from the respective proximal portion to the respective distal portion, substantially the entire length of the second leg member 104. The curvatures of the first leg member 102 and the second leg member 104 may substantially match, and the respective concavity/convexity of the first inner surface 108 and the second inner surface 112 may substantially match. The first and second leg member 102, 104 may bend and/or straighten as the surgical clip 100 closes, and the first and second inner surfaces 108, 112 may be approximated or contact in a closed configuration.

The first and/or second leg members 102, 104 are overmolded onto corresponding limbs of an elastic insert 170. Stated another way, the limbs of the elastic insert 170 are molded within the respective legs 102, 104 of the polymer clip 100 to form a substrate or spine for the overmolding process. For instance, the elastic insert 170 includes a first limb 172 and a second limb 174. The first limb 172 of the elastic insert 170 may have a curvature substantially corresponding to the curvature of the first leg member 102 of the polymer clip 100. The second limb 174 of the elastic insert 170 may have a curvature substantially corresponding to the curvature of the second leg member 104 of the polymer clip 100. The elastic insert 170 further comprises a hinge portion 106 connecting the first and second limbs 172, 174. The hinge portion 106 is free from the overmolded leg members 102, 104. The first leg member 102 is thus coupled to the second leg member 104 by the hinge portion 106 of the elastic insert 170.

The elastic insert 170 may further include at least one mechanical attachment 171, such as an oblong cutout or other shaped cutout, configured to secure the limbs of the elastic insert to the respective first leg member and/or the second leg member during the overmolding process. For example, the polymer material of the first and/or second leg members is able to enter the oblong cutouts of the elastic insert during the overmolding process, thus providing enhanced gripping of the elastic insert.

Exemplary materials for the first leg member 102 and the second leg member 104 include biocompatible polymers such as a homopolymer or co-polymer polyacetal, polyethylene terephthalate (PET), polybutylene terephthalate (PBT), polyoxymethylene, or other thermoplastic materials. All polymers possess some level of stress relaxation over time even when not exposed to elevated temperatures. The hinge portion 106 of the elastic insert 170 eliminates such stress relaxation. The elastic insert 170 may comprise a metal material with a high yield strength and no tendency to relax over time. Examples of such a metal material of the elastic insert include steel, stainless steel, nitinol, titanium, or various alloys of these base materials. These metal materials offer different yield strengths depending on the composition manufacturing process and heat treatment. In some implementations, the elastic insert 170 is formed of a single piece of metal material. In other implementations, for reasons of improved manufacturability and/or material cost, it should be appreciated that alternative concepts may be considered that modify the geometry of the hinge to improve stress concentrations, thereby dispersing stresses evenly throughout the hinge portion.

Accordingly, the hinge portion 106 of the elastic insert is made of an elastic material and may be flexible. The binge portion 106 material may also have a large yield strength (referred in Table 1 as “Minimum Tensile Strength”). Examples of potential material options for a stainless steel insert are shown in Table 1 below.

TABLE 1
Examples of Stainless Steel Material Options
Stainless Steels

| | | | | Minimum | Modulus | Modulus | | |
|---|---|---|---|---|---|---|---|---|
| | | | | Tensile | of | in | | |
| | Commercially | | | Strength | Elasticity | Torsion | Maximum | |
| | Available | Nominal | Density | (psi × | (E)(psi × | (G)(psi × | Operating | Description & |
| Material | Specification | Chemistry | (lb/in3) | 10³) | 10⁶) | 10⁶) | Temperature | Primary Use |
| Stainless | AMS 5678 | Cr 16.0- | 0.28 | Cond. | 29.5 | 11 | 600° F. | Improved |
| Steel 17- | | 18.0%, | | CH900: | | | | strength over |
| 7 PH | | Ni 6.5- | | 230-343 | | | | 300 Series |
| | | 7.7%, | | | | | | stainless |
| | | Al 0.75- | | | | | | steels with |
| | | 1.5% | | | | | | similar heat |
| | | | | | | | | and corrosion |
| | | | | | | | | resistance. |
| | | | | | | | | Slightly |
| | | | | | | | | magnetic. |
| | | | | | | | | Material is |
| | | | | | | | | precipitation |
| | | | | | | | | hardened |
| | | | | | | | | after spring |
| | | | | | | | | fabrication to |
| | | | | | | | | achieve |
| | | | | | | | | desired |
| | | | | | | | | properties. |
| Stainless | ASTM A313 | Cr 16.5- | 0.29 | 125-245 | 28 | 10 | 550° F. | General heat |
| Steel | | 18.0%, | | | | | | resistant |
| 316 | | Ni 10.5- | | | | | | spring wire |
| | | 13.5%, | | | | | | and better |
| | | Mo 2.0- | | | | | | corrosion |
| | | 2.5% | | | | | | resistance |
| | | | | | | | | than Type |
| | | | | | | | | 302/204. |
| | | | | | | | | Slightly |
| | | | | | | | | magnetic. |
| | | | | | | | | Often used in |
| | | | | | | | | marine |
| | | | | | | | | applications, |
| | | | | | | | | resists |
| | | | | | | | | chloride |
| | | | | | | | | attack. |
| Stainless | ASTM A313 | Cr 17.0- | 0.29 | 130-325 | 28 | 10 | 550° F. | General |
| Steel | | 20.0%, | | | | | | purpose |
| 302/304 | | Ni 8.0- | | | | | | corrosion and |
| | | 10.5% | | | | | | heat resistant |
| | | | | | | | | spring wire. |
| | | | | | | | | Slightly |
| | | | | | | | | magnetic. |
| | | | | | | | | Type 304 has |
| | | | | | | | | less carbon |
| | | | | | | | | than Type |
| | | | | | | | | 302, however |
| | | | | | | | | considered |
| | | | | | | | | commercially |
| | | | | | | | | equivalent for |
| | | | | | | | | stock |
| | | | | | | | | springs. |

The elastic insert 170 allows the surgical clip 100 to maintain stiffness and spring functionality at the hinge portion 106 over the lifespan of the surgical clip 100. Thus, the surgical clip 100 is able to minimize plastic deformation and maintain its boss-to-boss distance for the shelf-life of the safety clip 100. Furthermore, providing the hinge portion 106 with a material that is more resilient to high stresses and does not have stress relaxation over time eliminates the need to load the clip into a clip applier with the jaw members in a closed or partially closed configuration, where a constant force on the back of the clip hinge would be required to maintain the clip in an open configuration. Thus, the elastic insert 170 and hinge portion 106 provides improved operational efficiency of the surgical clip 100.

During use, for instance, the ability to load clips without removing the applier from the target vessel/tissue improves operational efficiency by eliminating the need to reposition the applier on the vessel/tissue after loading the next clip. Additionally, the elastic insert 170 and hinge portion 106 help reduce inadvertent damage to vessels/structures. During use, for instance, enabling the clip to be reloaded into a clip applier with the leg members in an open configuration eliminates the potential misuse of closing the leg members of the clip on vessels/structures while loading the clip into the applier, thus avoiding the potential to do unintended damage to the vessel/tissue.

The surgical clip 100 may be continuously compressed from an initial or open state (i.e., as manufactured) to a final or closed state (i.e., the first and second leg members are latched together). The leg members 102, 104 of the surgical clip 100 may also be partially compressed. According to one aspect, the leg members 102, 104 may be partially compressed to a loaded state. In the loaded state, the surgical clip 100 is partially compressed when loaded in the jaws of the applier. In this loaded state, it is important to consider the compressed hinge force, since there is a minimum force necessary to ensure the clip remains retained in the mating features between the clip and applier jaws to minimize the likelihood of the clip failing out during manipulation around vessels/structures and adjacent tissue.

Conventional clips that do not have stress relaxation capabilities have a retention force in the loaded state of 0.1-0.2 lbf (0.4-0.9 N) when manufactured. However, they lack this retention force after being stored in a closed or partially closed state. A target preload of the surgical clip 100 of the present invention in the loaded state against the jaws of an applier is a minimum of 0.1 lbf.

According to another aspect, the leg members 102, 104 may be partially compressed to a stored state, which is the configuration that the clip will be stored in prior to use on a vessel/tissue. In the stored state, the surgical clip 100 is partially compressed when loaded in the shaft of the applier, prior to it being loaded in the jaws of the applier. In most cases, this stored state is when the latch features are touching but are not positively engaged, which is also the minimum height the clip can be stored in. The stress in the hinge portion is elevated while the clip is in the stored state. If plastic strain or stress is observed in this stored state, then the clip will not fully return to its original manufactured state. Thus, with regard to the stored state strength, permanent plastic deformation of the clip is avoided by selecting a material of the elastic insert that does not exceed the allowable strain and stress in the hinge portion.

The biasing spring force of the elastic insert 170 at the hinge portion 106 acting to open the surgical clip 100 during jaw closure or clip latching may be greater than conventional polymer ligating clips, and thus an increase in applier jaw strength helps to manage those forces and prevent plastic deformation of the jaws during latching. If plastic deformation occurs in the jaws of the device, then the ability of the applier to repeatedly latch the clip will be negatively impacted. This consideration is important due to the limited space available for additional material to increase jaw strength, particularly in multi-fire design configurations where material must be removed to provide a channel for the clip to be loaded in the applier between firings. The hinge force of conventional polymer clips is approximately 0.5 lbf. The target hinge force of the surgical clip 100 of the present invention during closure is not to exceed 0.5 lbf. An increase in hinge force requires additional jaw strength considerations or a reduction in latch force to offset the additional hinge force.

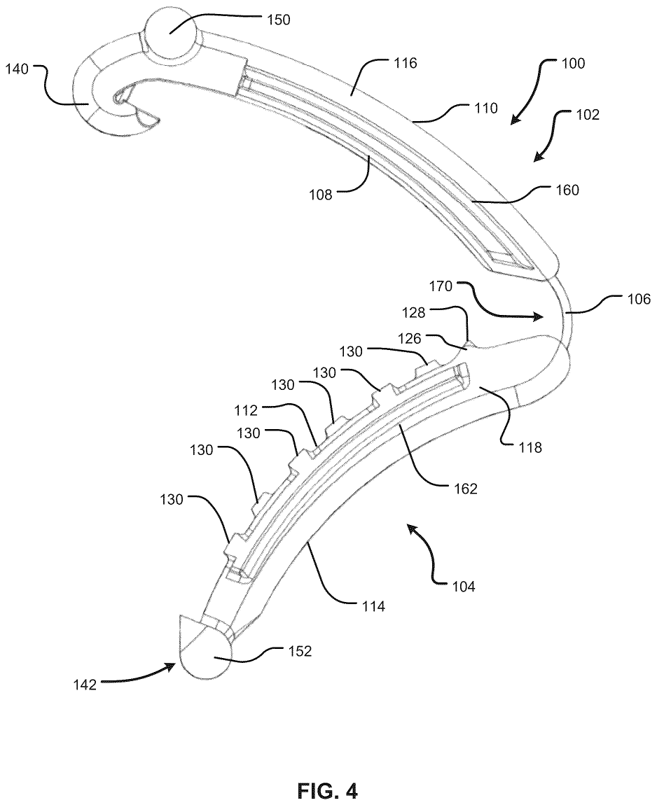

The surgical clip 100 may further have a convex portion or heel 126 on a proximal portion of the second leg member 104. The heel 126 may have a convex curvature having a smaller radius of curvature than the remaining length of the second inner surface 112, providing the heel 126 with increased convexity. The heel 126 may include at least one protrusion or tooth 128 to increase securement of the tissue. For example, the heel 126 may include a single tooth 128 extending substantially the width of the heel 126. As illustrated in FIG. 4, the at least one tooth 128 may be angled proximally from the heel 126 toward the hinge member 106 to provide a counterforce to further prevent pull-off of the surgical clip 100.

The surgical clip 100 may also include a latching mechanism having one or more latching elements. For example, the first leg member 102 may transition to a hook member 140 at its distal portion, and the second leg member 104 may transition to a complementary grooved tip member 142 at its distal portion. A distal portion of the hook member 140 may curve inwardly and point generally toward the hinge member 106. The hook member 140 may have one or more transverse beveled surfaces and a concave inner surface which merges with the first inner surface 108 to define a latching recess. The tip member 142 may define a V-shaped groove or slot configured to receive the beveled surfaces of the hook member 140, as the hook member 140 opens or deflects around the tip member 142 and/or the second leg member 104 compresses. The hook member 140 and the tip member 142 may engage to form the latching mechanism. For example, the latching recess may receive the tip member 142 in the course of compressing the surgical clip 100 into the closed configuration when secured position around a vessel or other tissue.

The leg members 102, 104 may include one or more boss members along the length to engage jaws of the clip applier. For example, the first leg member 102 may include one or more first boss members 150 protruding perpendicular to opposing side surfaces adjacent to the distal portion of the first leg member 102 and immediately proximal of the hook member 140. In the illustrated example of the surgical clip 100, the one or more first boss members 150 may be cylindrical and project outwardly beyond each of the side surfaces 116 of first leg member 102. The first boss members 150 may include a bridge section extending the width of the first leg member 102 and joining opposing first boss members 150 extending laterally of the side surfaces 116. The second leg member 104 may also include one or more second boss members 152 at the distal portion spaced apart by the slot of the tip member 142. The second boss members 152 may be cylindrical and protrude perpendicularly to opposing side surfaces 118 of the second leg member 104, extending longitudinally forward beyond the point of tip member 142 and outwardly beyond the side surfaces 118 of second leg member 104. The jaws of the clip applier may engage the boss members 150, 152 and pivot the leg members 102, 104 about the hinge member 106 to compress the surgical clip 100 into the closed and/or latched configuration around a vessel.

As further shown in the embodiment of FIGS. 2-6, the surgical clip 100 may include a plurality of teeth 130. The teeth 130 may be substantially rigid, such that the teeth 130 do not substantially deflect when engaging tissue. The teeth 130 may be positioned out-board relative to the surgical clip 100. As used herein, the term “out-board” refers to the positioning of the teeth 130 laterally of the inner surface 112. For example, the teeth 130 may extend inwardly from at least one wing member or lateral protrusion 162 extending perpendicularly from at least one of the side surfaces 118. The teeth 130 may have a substantially flat outer lateral side surface and a substantially flat inner side surface (defined by the exposed surface extending perpendicular from the inner surface 112). The substantially flat inner side surface of the teeth 130 may make a flush interface or minimal gap with the side surfaces 116 of the first leg member 102. The teeth 130 may further have a substantially flat inner surface extending substantially parallel to the second inner surface 112 to reduce trauma on the ligated tissue.

The teeth 130 may form a first row of teeth 130 extending from a first lateral protrusion 162 integrated into a first side surface 118 and a second row of teeth 130 extending from a second lateral protrusion 162 integrated into a second side surface 118. The teeth 130 may be positioned to clear the first inner surface 108 and extend along the side surfaces 116 of the first leg member 102 to enable the surgical clip 100 to close with minimal or no gap between the inner surfaces 108, 112 ensuring effective closure of small vessels.

The first and second rows of teeth 130 may be configured to receive the first inner surface 108 therebetween in the closed configuration. Thus, in the closed configuration, the teeth 130 may extend from the lateral protrusions 162 past the first inner surfaces 108, and along or parallel to one of the first side surfaces 116. The teeth 130 may be sufficiently spaced apart from each other along the longitudinal axis of the second leg member 104 to prevent pinching tissue between adjacent teeth 130. The teeth 130 of the first and second rows may be staggered, alternating along the longitudinal axis of the inner surface 112.

The configuration of the teeth 130 may provide a favorable tortuous engagement of tissue with closely approximated tissue engaging surfaces. The larger size of the teeth 130 may improve tissue retention and prevent the tissue from slipping out of the surgical clip.

The teeth 130 may further be easy to mold and not interfere with clip appliers. Thus, due to the out-board teeth of the second leg member 104 and the absence of teeth on the first leg member 102, the first inner surface 108 and the second inner surface 112 may be substantially smooth (e.g., substantially without teeth) for substantially the entire length or majority of the length of the first and second leg members 102, 104, with the exception for example of the tooth 128 on the heel 126. For example, as illustrated, the first inner surface 108 may be completely without teeth, and the second inner surface 112 may be without teeth for the majority of its length.

The first leg member 102 may have at least one wing member or lateral protrusion 160 extending laterally from one of the first side surfaces 116. For example, the at least one lateral protrusion 160 may include a first lateral protrusion extending from a first side surface 116 and a second lateral protrusion extending from a second side surface 116. The lateral protrusions 160 may reinforce and stabilize the leg member 102 by increasing torsional stiffness. For example, the lateral protrusions 160 may enable the first leg member 102 to be longer without compromising the torsional and/or tissue retention strength of the surgical clip 100. The lateral protrusions 160 may extend longitudinally for at least half of the length of the first leg member 102. In some embodiments, the lateral protrusions 160 may extend longitudinally for at least two-thirds of the length of the first leg member 102. However, the lateral protrusion 160 may not extend the entire length of the first leg member 102. Thus, the lateral protrusions 160 may have a distal end that is proximal of the at least one first boss member 150 to prevent interference with the clip applier interface and/or a proximal end that is distal of the hinge member 106 to maintain the flexibility of the hinge member, reducing the pivoting stiffness of the leg members 102, 104. Furthermore, the width of the surgical clip 100 at the lateral protrusions 160 may be narrower than the width of the surgical clip 100 at the first boss members 150.

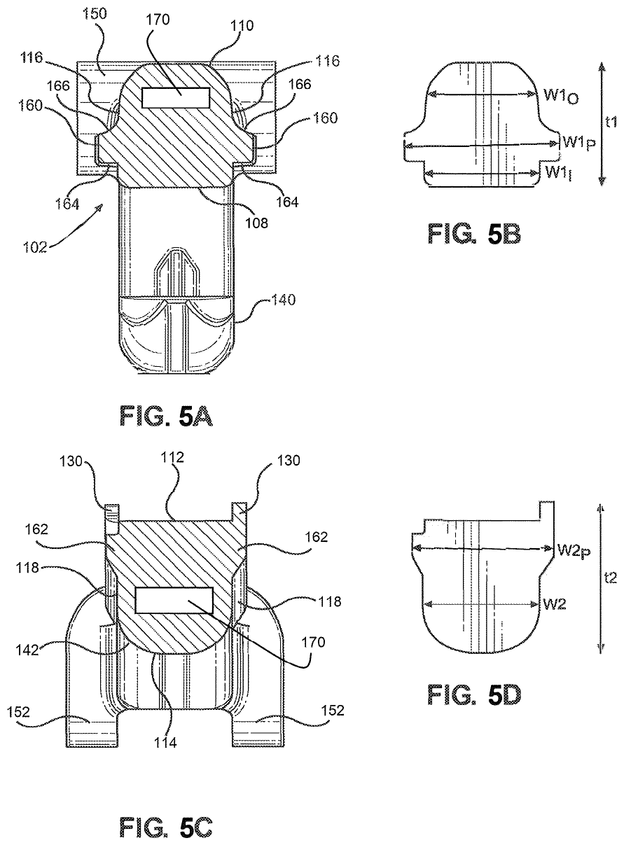

As illustrated in the cutaway of the first leg member 102 in FIG. 5A, the protrusion 160 may have an inner surface 164 spaced vertically from the first inner surface 108 and an outer surface 166 spaced vertically from the first outer surface 110. The spacing of the inner surface 164 of the protrusion from the first inner surface 108 may prevent interference with tissue ligation and allow the teeth 130 to receive the inner surface 108 therebetween. The inner surface 164 may be substantially flat and extend substantially perpendicular from the side surface 116 to further reduce any interference with the teeth 130 and/or provide a surface for the teeth 130 to engage in the closed configuration. The outer surface 166 may be angled, tapered, and/or curved (concave or convex) to increase the stability of the protrusion 160 and facilitate manufacturing. As illustrated, the side surface of the protrusion 160 may be free and substantially flat. The spacing of the lateral protrusion 160 from the first inner surface 108 and first outer surface 110 may further optimize the reinforcement of the surgical clip 100.

As further illustrated in the cut-away view of FIG. 5A and the cross-section of FIG. 5B, the first leg member 102 may have a protrusion width (W1_{P}) defined by side surfaces of opposing protrusions 160. The protrusion width (W1_{P}) may be greater than a first inner width (W1_{I}) defined by the dimension between the side surfaces 116 positioned between the protrusion 160 and the first inner surface 108. The protrusion width (W1_{P}) may also be greater than a first outer width (W1_{O}) defined by the dimension between the side surfaces 116 and positioned between the protrusion 160 and to the first outer surface 110. The protrusion width (W1ₚ) may further be greater than a first thickness (t1) defined by the dimension between the first inner surface 108 and the first outer surface 110. The protrusions 160 may extend for at least half of the length of the first leg member 102, thus the protrusion width (W1_{P}) may be greater than the first thickness (t₁), first inner width (W1_{I}), and/or the first outer width (W1_{O}) for at least half of the length of the first leg member 102.

In embodiments where the protrusion 160 extends for at least two-thirds of the length of the first leg member 102, the protrusion width (W1_{P}) may be greater than the first thickness (11), first inner width (W1_{I}), and/or the first outer width (W1_{O}) for at least two-third of the length of the first leg member 102. The distal end of the at least one lateral protrusion 160 may define a distal surface that extends substantially perpendicular to the first inner surface 108 and the first outer surface 110. The proximal end of the at least one lateral protrusion 160 may define a proximal surface that extends at an angle proximally relative to the first inner surface 108. The first leg member 102 may have substantially flat side surfaces 116 distal and proximal of the lateral protrusions 160 (e.g., between the lateral protrusion 160 and the first boss member 150 and/or between the lateral protrusion 160 and the hinge member 106). The outer surfaces 110, 114 of may have a rounded curvature extending between the respective side surfaces 116, 118 enhancing applier jaw strength and facilitating easier loading from a cartridge. An outer surface 122 of the hinge member 106 may have a similar rounded curvature. Each of the lateral protrusions 160 may themselves have a width from the respective side surface 116 no greater than a quarter of the first inner width (W1_{I}) and the first outer width (W1_{O}). FIGS. 2-6 illustrate a single lateral protrusion 160 extending from each side surface 116, which facilitates manufacturing of the surgical clip 100.

However, it is also contemplated that the surgical clip 100 may include a plurality of lateral protrusions 160 on each side surface 116 (e.g., longitudinally aligned and/or spaced apart) collectively extending the lengths as described herein.

As further illustrated in the cut-away view of FIG. 5C and the cross-section of FIG. 5D, the second leg member 104 may have a protrusion width (W2_{P}) defined by side surfaces of opposing protrusions 162. The protrusion width (W2_{P}) may be greater than a second width (W2) defined by the dimension between the side surfaces 118 and positioned between the protrusion 160 and to the second outer surface 114. The protrusion width (W2_{P}) may further be greater than a second thickness (12) defined by the dimension between the second inner surface 112 and the second outer surface 114. The protrusion 162 may extend for at least half of the length of the second leg member 104, thus the protrusion width (W2_{P}) may be greater than the second thickness (t2), and/or the second width (W2) for at least half of the length of the second leg member 104. In embodiments where the protrusion 162 extends for at least two-thirds of the length of the second leg member 104, the protrusion width (W2_{P}) may be greater than the second thickness (t2), and/or the second width (W2) for at least two-thirds of the length of the second leg member 104. The distal end of the at least one lateral protrusion 162 may defined a distal surface that extends substantially perpendicular to the first side surface 116, the second inner surface 110, and/or the second outer surface 110. The proximal end of the at least one lateral protrusion 162 may define a proximal surface that extends at an angle proximally relative to the second outer surface 114 and distally relative to the second inner surface 112. The second leg member 104 may have substantially flat side surfaces 118 distal and/or proximal of the lateral protrusions 162 (e.g., between the lateral protrusion 162 and the second boss member 152 and/or between the lateral protrusion 162 and the hinge member 106).

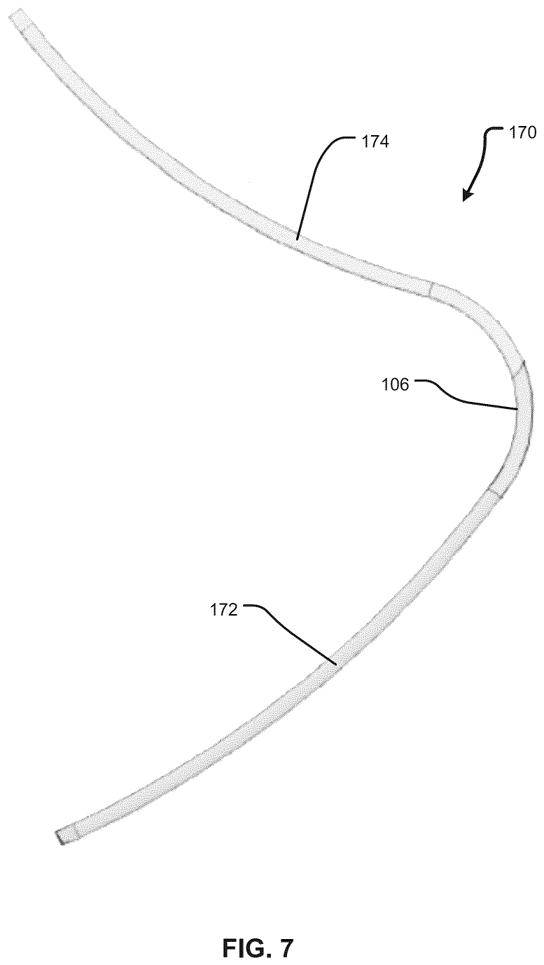

Additionally, design optimization helps to manage the design requirements of the hinge portion 106 of the surgical clip. For instance, the elastic insert 170 and the hinge portion 106 may have a uniform rectangular-shaped cross-section. Increasing the thickness of the elastic insert increases the stiffness, stress, and strain in the insert. Therefore, a thinner elastic insert may be preferable. However, if the elastic insert is too thin it will not provide enough preload in the aforementioned loaded state. It should be appreciated that alternative designs may be contemplated to increase the preload and stiffness of the elastic insert while managing stresses in the insert to meet design requirements. For example, FIG. 7 depicts the elastic insert 170 described above made of a unitary piece of metal including a uniform rectangular-shaped cross-section.

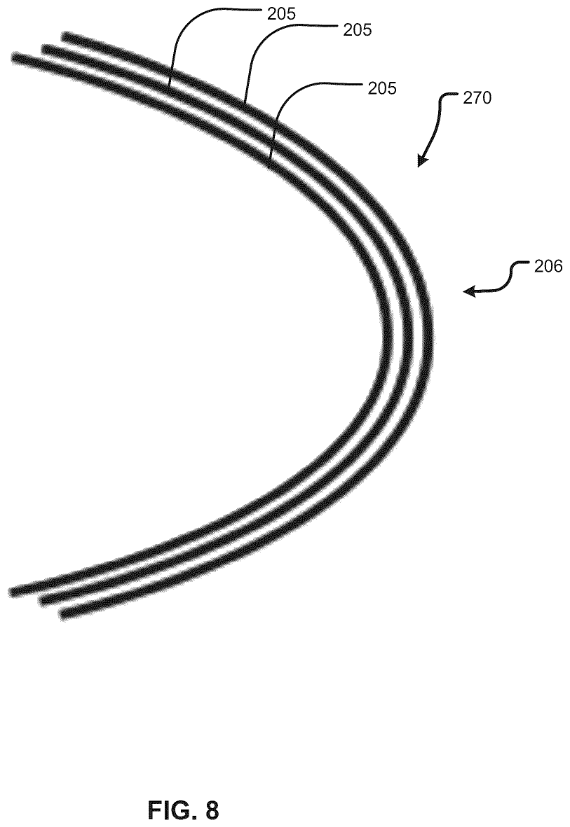

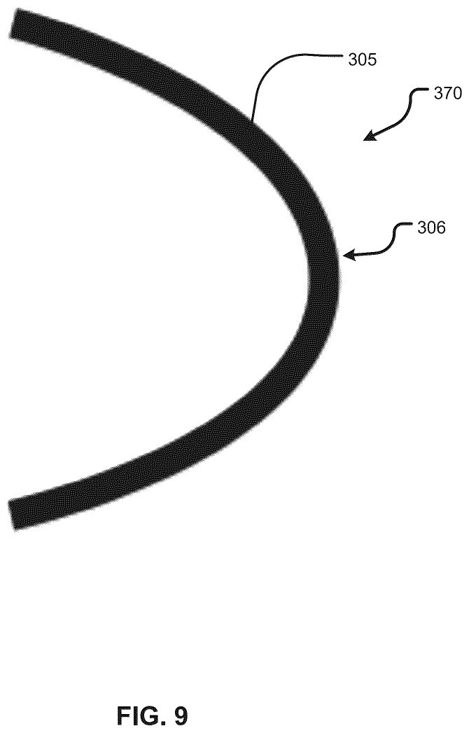

FIGS. 8 and 9 depicts an elastic insert 270, 370 with a laminate spine configuration, which involves stacking several thinner elastic inserts 205 (e.g., three thin laminate elastic layers 205 in FIG. 8 and one thick elastic layer 305 in FIG. 9). The elastic layers may be joined if necessary using another material such as a silicone, urethane or other suitable adhesive material. This configuration is especially useful to manage stress and strain along the length of the entire elastic insert 270. Further, each insert 270 includes a respective hinge portion 206 configured to prevent or minimize stress relaxation similar to the hinge portion 106 previously discussed above.

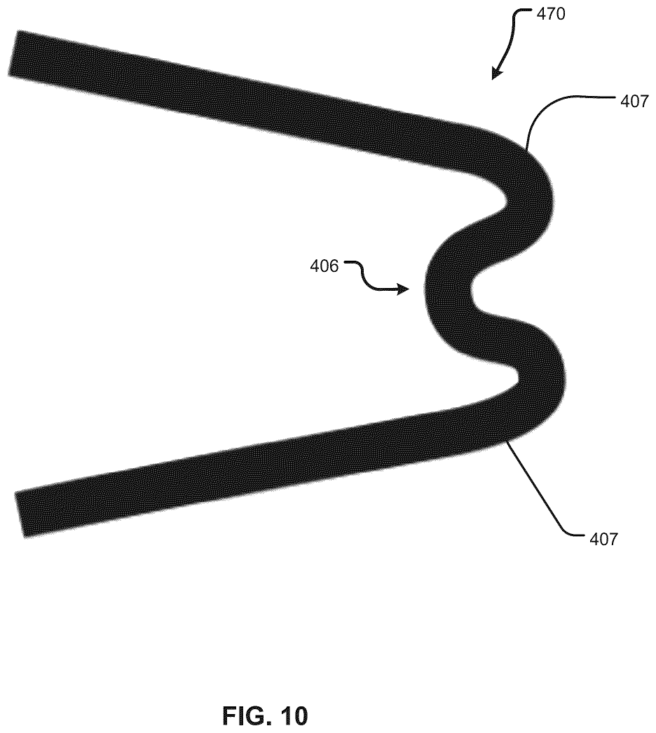

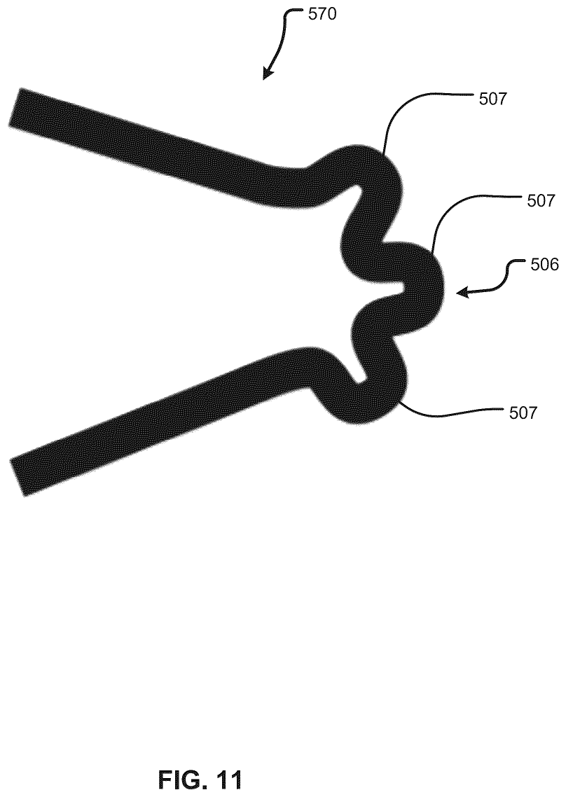

Further, FIGS. 10 and 11 depict an elastic insert 470, 570 with a wavy spine configuration. In this configuration, the elastic insert 470, 570 includes a plurality of bends 407, 507, which are preferably arc-shaped, at the respective hinge member 406, 506, and which are configured to spread the stress along the length of the elastic insert 470, 570, rather than having the stress concentrated at a single bend at the hinge member 406, 506. FIG. 10 depicts the elastic insert 470 with two bends 407 at the hinge member 406, while FIG. 10 depicts the elastic insert 570 with three bends 507 at the hinge member 506. However, FIGS. 10 and 11 are only illustrative and any number of bends that are capable to avoid the concentration of stress at the hinge member are contemplated.

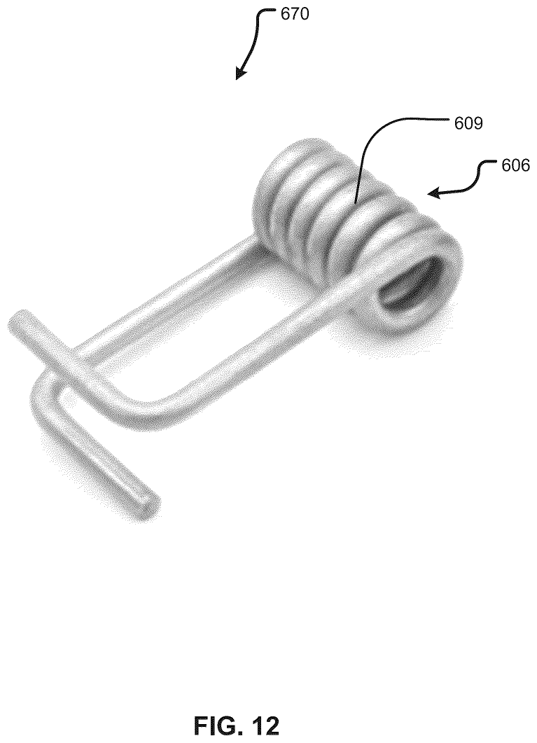

FIG. 12 depicts an elastic insert 670 with a coil configuration. In this configuration, the elastic insert includes a coil 609 at the hinge member 607. The coil 609, in this manner, increases the allowable angular deflection the surgical clip 100 may undergo before reaching plastic deformation, improving the longevity and durability of the clip 100.

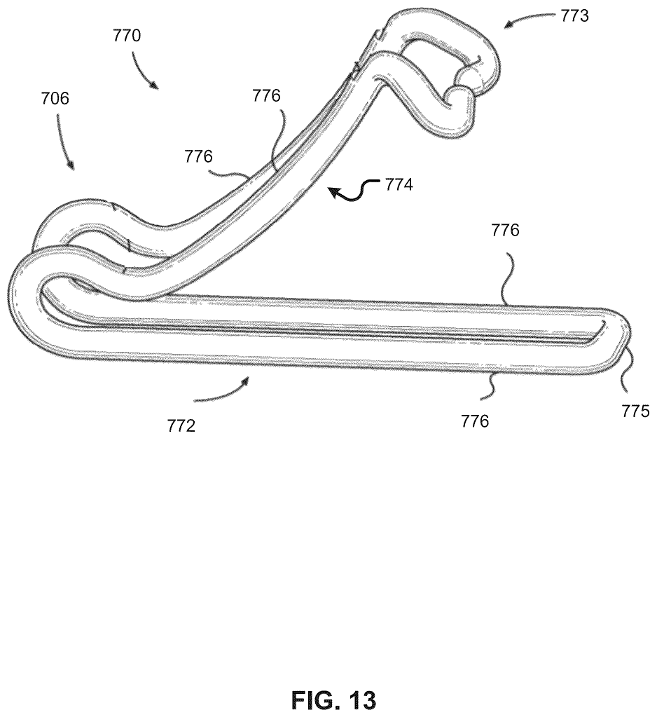

Finally, FIG. 13 depicts the elastic insert 770 with a wire configuration. Instead of a flat piece of metal, this configuration uses metal wire in forming the elastic insert 770 of the surgical clip 100. This elastic insert 770 may include a first limb 774 and a second limb 772 joined by the hinge portion 706. Thus, this elastic insert 770 may be formed of a single, unitary wire body having a first free end portion 773 and a second free end portion 775. The elastic insert 770 in this configuration may have a cross-section including one of more of circular, round, ellipsoid, rectangular with rounded corners, square, rectangular, and/or polygonal segments. For example, as depicted in FIG. 13, the elastic insert 770 may have a round and/or circular cross-section through substantially the entire length of the wire body. In some embodiments, the wire body may extend continuously between the first free end portion 773 and the second free end portion 775. In this way, round and/or the rectangular segments with rounded corners may advantageously reduce stress localizations on compressed tissue.

As further depicted in FIG. 13, the first limb 774 and the second limb 772 may include first and second segments 776 of the wire body. The first and second segments 776 may be spaced along their lengths. The first free end portion 773 and the second free end portion 775 may be positioned on the distal ends first and second limbs 772, 774 and may be moveable relative to each other through the deflection of the first and second segments 776.

The various embodiments of the surgical clips of the present invention may be made of any suitable size and may be applied to any number of tissues, such as blood vessels, lymph nodes, nerves, cystic ducts, and cardiac tissue. The various embodiments of the surgical clips may be constructed from any suitable biocompatible material, such as metals and polymers. However, the present invention is particularly suitable for practice with polymeric clips.

What is claimed is:

1. A surgical clip comprising:
a first leg member including a first proximal portion, a first distal portion, and a first inner surface;
a second leg member including a second proximal portion, a second distal portion, and a second inner surface; and
an elastic insert having a first limb attached to the first leg member, a second limb attached to the second leg member, and a hinge portion configured to pivotably bias the first and second leg members toward an open configuration.

2. The surgical clip according to claim 1, wherein the hinge portion is further configured to prevent or resist stress relaxation when the first and second leg members are in a closed or partially closed configuration in which the hinge portion is compressed.

3. The surgical clip according to claim 1, wherein the hinge portion is free of the first and second leg members, such that the first proximal portion of the first leg member is spaced apart from the second proximal portion of the second leg member when the first and second leg members are in the open configuration.

4. The surgical clip according to claim 1, wherein the first leg member includes at least one first boss member configured to engage a first jaw of a clip applier, and wherein the second leg member includes at least one second boss member configured to engage a second jaw of the clip applier.

5. The surgical clip according to claim 4, wherein the hinge portion is configured to maintain a distance between the at least one first boss member of the first leg member and the at least one second boss member of the second leg member when the first and second leg members are in the open configuration after the first and second leg members have been in a closed configuration for a period of time.

6. The surgical clip according to claim 1, wherein the hinge portion is made of a material having at least one of a yield strength and an allowable strain greater than that of the first leg member and/or the second leg member.

7. The surgical clip according to claim 6, wherein the first and second leg members are formed of a biocompatible polymer material.

8. The surgical clip according to claim 1, wherein the first and/or second limbs of the elastic insert form a substrate for an overmolding process.

9. The surgical clip according to claim 8, wherein the first leg member is overmolded onto the first limb of the elastic insert and/or the second leg member is overmolded onto the second limb of the elastic insert.

10. The surgical clip according to claim 1, wherein the elastic insert comprises at least one mechanical attachment configured to secure the elastic insert to the first leg member and/or the second leg member.

11. The surgical clip according to claim 10, wherein the at least one mechanical attachment comprises a hole.

12. The surgical clip according to claim 1, wherein the elastic insert has a rectangular-shaped cross-section.

13. The surgical clip according to claim 1, wherein the elastic insert is formed of a single piece of metal material.

14. The surgical clip according to claim 1, wherein the hinge portion has an arc-shaped bend.

15. The surgical clip according to claim 1, wherein the hinge portion includes a coil spring.

16. The surgical clip according to claim 1, wherein the first inner surface of the first leg member has a first curvature, and the second inner surface of the second leg member has a second curvature.

17. The surgical clip according to claim 16, wherein the first curvature of the first inner surface of the first leg member matches the second curvature of the second inner surface of the second leg member when the first and second leg members are in a closed configuration.

18. The surgical clip according to claim 17, wherein the first limb of the elastic insert has a first limb curvature approximately matching the first curvature of the first inner surface of the first leg member, and wherein the second limb of the elastic insert has a second limb curvature approximately matching the second curvature of the second inner surface of the second leg member.

19. The surgical clip according to claim 1, further comprising a hook member on the first distal portion of the first leg member, and a tip member on the second distal portion of the second leg member, wherein the hook member is configured to receive the tip member to retain the surgical clip in a closed configuration.

20. The surgical clip according to claim 1, wherein the elastic insert comprises a circular cross-section.
